(19) United States
(12) Patent Application Publication
(10) Pub. No.: US 2005/0232973 A1
(43) Pub. Date: Oct. 20, 2005

(54) Systems and methods for preparation of pharmaceutical dosage using compositions containing aqueous vesicles

(76) Inventor: Makarand Gore, Corvallis, OR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/827,484

(22) Filed: Apr. 19, 2004

(51) Int. Cl. A61K 9/127; A61K 47/00; B41J 2/01

(52) U.S. Cl. 424/439; 424/450; 347/1

(57) ABSTRACT

A jettable solution includes a plurality of vesicles, and a pharmaceutical payload associated with the vesicles.

### BACKGROUND

Traditional oral dosage drug formulations include both active pharmaceutical ingredients (API) and inactive ingredients. The inactive ingredients, also called excipients, are components of the final formulation of a drug that are not considered active pharmaceutical ingredients (API) in that they do not directly affect the consumer in the desired medicinal manner.

Traditional oral dosage forms have several inactive ingredients. Among the traditional inactive ingredients included in oral dosage forms are binders that hold the tablet together, coatings configured to mask an unpleasant taste, disintegrants configured to make the tablet break apart when consumed, enteric coatings, fillers that assure sufficient material is available to properly fill a dosage form, enhancers configured to increase stability of the active ingredients, preservatives aimed at preventing microbial growth, and the like.

Additionally, a number of desirable properties may be attributed to pharmaceuticals through the inclusion of liposomes. More specifically, liposome based pharmaceutical delivery provides high solubility, high absorption, and improved pharmacokinetics.

Traditionally, the formation of an oral dose drug often included combining a desired pharmaceutical product with a specified combination of materials designed to control the release rate of the API when consumed. While the traditional method is effective for a number of soluble drugs, there are a number of highly insoluble drugs that are not well suited to sustained or controlled delivery. The formulation of these highly insoluble APIs into controlled or modified-release dosage forms using traditional formulation methods is both expensive and challenging due to the APIs insolubility and unknown stability. Moreover, the challenges of formulating a modified-release dosage form are increased when implementing a liposome based delivery system.

### SUMMARY

A jettable solution includes a plurality of vesicles, and a pharmaceutical payload associated with the vesicles.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is a simple block diagram illustrating a system that may be used to deposit an aqueous vesicle containing a pharmaceutical product, according to one exemplary embodiment.

FIG. 2 is a simplified structural diagram illustrating the structure of a lipid, according to one exemplary embodiment.

FIG. 3 is a magnified view illustrating a unilamellar liposome vesicle, according to one exemplary embodiment.

FIG. 4 is a magnified view of a multilamellar liposome vesicle including a pharmaceutical payload, according to one exemplary embodiment.

FIG. 5 is a flow chart illustrating a method for forming an aqueous vesicle configured to house a pharmaceutical payload, according to one exemplary embodiment.

FIG. 6 is a simple block diagram illustrating a method for dispensing an aqueous vesicle configured to house a pharmaceutical payload, according to one exemplary embodiment.

Throughout the drawings, identical reference numbers designate similar, but not necessarily identical, elements.

### DETAILED DESCRIPTION

A number of exemplary systems and methods for producing an ink jettable aqueous vesicle containing a pharmaceutical payload are disclosed herein. More specifically, a jettable aqueous vesicle pharmaceutical is disclosed that is formed from a number of liposomes containing pharmaceutical payloads, which may include immiscible pharmaceuticals. Moreover, an exemplary method for forming and precisely metering the jettable aqueous vesicle pharmaceutical with an inkjet material dispenser to form an oral dosage form is disclosed herein.

As used in the present specification and the appended claim, the term “edible” is meant to be understood broadly as any composition that is suitable for human consumption and is non-toxic. Similarly, the phrase “suitable for human consumption” is meant to be understood as any substance that complies with applicable standards such as food, drug, and cosmetic (FD&C) regulations in the United States and/or Eurocontrol experimental centre (E.E.C.) standards in the European Union. Additionally, the term “ink” is meant to be understood broadly as meaning any jeftable fluid configured to be selectively emitted from an inkjet dispenser, regardless of whether the jettable fluid contains a dye or any other colorant. The term “jettable” is meant to be understood both in the present specification and in the appended claims as any material that has properties sufficient to allow the material to be selectively deposited by any digitally addressable inkjet material dispenser.

Additionally, in the present specification and in the appended claims, the term “liposome” is meant to be understood broadly as including any microscopic globule of lipids configured to enclose a desired material. Additionally, the term “pharmacokinetics” or “PK” is meant to be understood as referring to the metabolism and action of a drug, with particular emphasis on the time required for absorption, duration of action, distribution in the body, and excretion. Moreover the term “sonicate” is meant to be understood as a process for exposing a suspension of cells, pharmaceuticals, and/or liposomes to the disruptive effect of the energy of high frequency sound wave.

In the following description, for purposes of explanation, numerous specific details are set forth in order to provide a thorough understanding of the present system and method for forming and controllably dispensing aqueous vesicles containing a pharmaceutical component will be apparent, however, to one skilled in the art, that the present method may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

Exemplary Structure

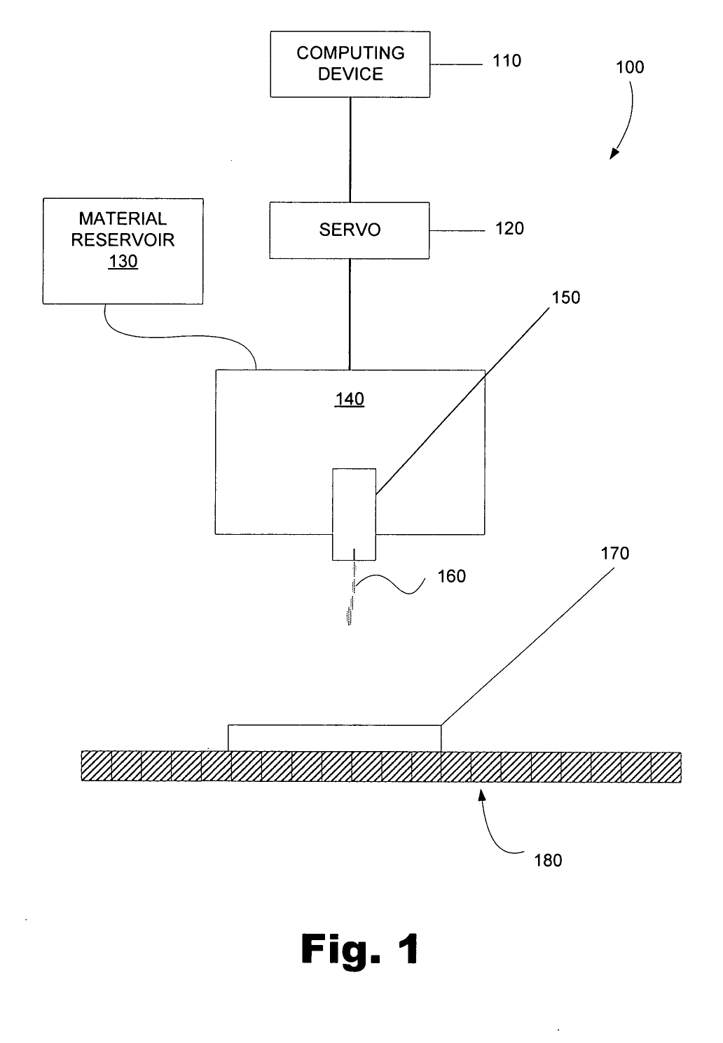

FIG. 1 illustrates an exemplary formulation system (100) that may be used to apply an aqueous vesicle pharmaceutical (160) to an edible structure (170) according to one exemplary embodiment. As shown in FIG. 1, the present system includes a computing device (110) controllably coupled through a servo mechanism (120) to a moveable carriage (140) having an inkjet dispenser (150) disposed thereon. A material reservoir (130) is also illustrated as fluidly coupled to the inkjet material dispenser (150). Moreover, a substrate (180) is located adjacent to the inkjet dispenser (150) having an edible structure (170) disposed thereon. The edible structure (170) is configured to receive an aqueous vesicle pharmaceutical (160). The above-mentioned components of the present formulation system (100) will now be described in further detail below.

The computing device (110) that is controllably coupled to the servo mechanism (120), as shown in FIG. 1, controls the selective deposition of the aqueous vesicle pharmaceutical (160) onto the edible structure. According to one exemplary embodiment, a digital representation of the desired deposition of aqueous vesicle pharmaceutical (160) may be generated on an application hosted by the computing device (110). The generated representation may then be converted into servo instructions that are housed in a processor readable media (not shown). When accessed by the computing device (110), the instructions housed in the processor readable media are used to control the servo mechanisms (120) as well as the movable carriage (140) and the inkjet dispenser (150), causing them to selectively deposit the aqueous vesicle pharmaceutical (160). The computing device (110) illustrated in FIG. 1 may be, but is in no way limited to, a workstation, a personal computer, a laptop, a personal digital assistant (PDA), or any other processor containing device.

The moveable carriage (140) of the present formulation system (100) illustrated in FIG. 1 is a moveable material dispenser that may include any number of inkjet material dispensers (150) configured to dispense the present aqueous vesicle pharmaceutical (160). The moveable carriage (140) may be controlled by a computing device (110) and may be controllably moved by, for example, a shaft system, a belt system, a chain system, etc. making up the servo mechanism (120). As the moveable carriage (140) operates, the computing device (110) may inform a user of operating conditions as well as provide the user with a user interface.

As a desired quantity of the aqueous vesicle pharmaceutical (160) is printed, the computing device (110) may controllably position the moveable carriage (140) and direct one or more of the inkjet dispensers (150) to selectively dispense the aqueous vesicle pharmaceutical at predetermined locations on the edible structure (170) as digitally addressed drops. The inkjet material dispensers (150) used by the present formulation system (100) may be any type of inkjet dispenser configured to perform the present method including, but in no way limited to, thermally actuated inkjet dispensers, mechanically actuated inkjet dispensers, electrostatically actuated inkjet dispensers, magnetically actuated dispensers, piezo-electrically actuated inkjet dispensers, continuous inkjet dispensers, etc.

The material reservoir (130) that is fluidly coupled to the inkjet material dispenser (150) houses the aqueous vesicle pharmaceutical (160) prior to printing. The material reservoir (130) may be any sterilizeable container configured to hermetically seal the aqueous vesicle pharmaceutical (160) prior to printing and may be constructed of any number of materials including, but in no way limited to, metals, plastics, composites, ceramics, or appropriate combinations thereof.

FIG. 1 also illustrates the components of the present system that facilitate reception of the aqueous vesicle pharmaceutical (160) and the edible structure (170). As shown in FIG. 1, a substrate (180) may receive and/or positionally secure an edible structure (170) during a printing operation. The edible structure (170) configured to receive the aqueous vesicle pharmaceutical (160) may be any number of edible substrates. According to one exemplary embodiment, the edible structure (170) includes, but is in no way limited to, polymeric and/or paper organic film formers. Non-limiting examples of such substrates include starch (natural and chemically modified), glycerin based sheets with or without a releasable backing, and the like; proteins such as gelatin, wheat gluten, and the like; cellulose derivatives such as hydroxypropylmethylcellulose, methocel, and the like; other polysaccharides such as pectin, xanthan gum, guar gum, algin, pullulan (an extracellular water-soluble microbial polysaccharide produced by different strains of Aureobasidium pullulans), and the like; sorbitol; seaweed; synthetic polymers such as polyvinyl alcohol, polymethylvinylether (PVME), poly-(2-ethyl 2-oxazoline), polyvinylpyrrolidone, and the like. Further examples of edible delivery substrates are those that are based on milk proteins, rice paper, potato wafer sheets, and films made from restructured fruits and vegetables. It should be understood that one or more of the above listed substrate materials, as well as other substrate materials, may be used in combination in some embodiments. The formation and composition of the aqueous vesicle pharmaceutical (160) will now be described in detail below.

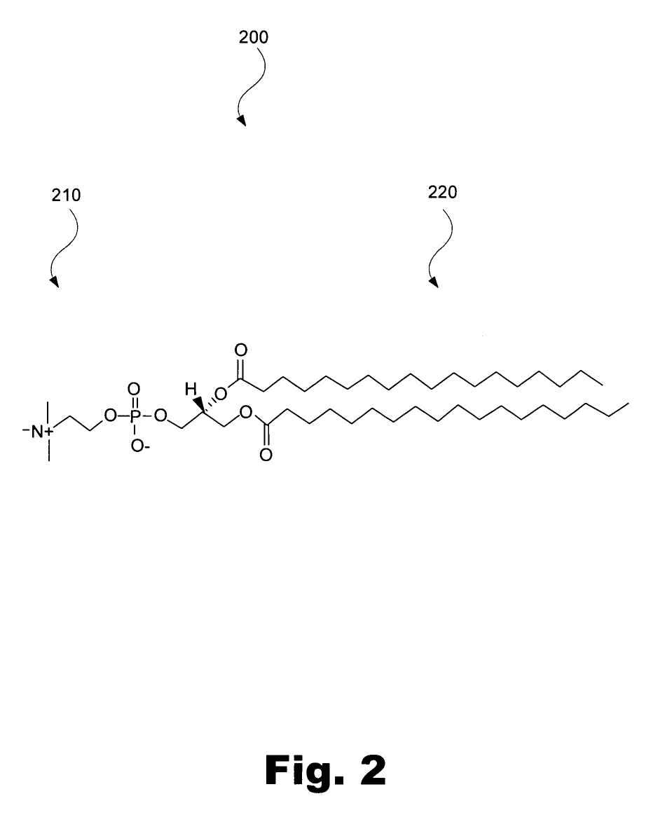

According to one exemplary embodiment, the aqueous vesicle pharmaceutical (160) includes vesicle forming lipids (200) as illustrated in FIG. 2. Lipids (200) are substances that are soluble in organic solvents but are only sparingly soluble or insoluble in water. Additionally, lipids (200) are generally classified according to their backbone structure, and include fatty acids, triacylglycerols, glycerophospholipids, sphingolipids, steroids, and the like. As illustrated in FIG. 2, vesicle-forming lipids (200) usually include two nonpolar “tail” groups (220) attached to a polar “head” group (210). According to one exemplary embodiment, when a number of the vesicle forming lipids (200) illustrated in FIG. 2 are introduced into an aqueous media, hydrophobic and Van der Waals forces drive the molecules to organize themselves into a “bilayer” or a sheet-like structure two molecules deep and oriented in such a way that each non-polar end interacts with another non-polar end and the polar ends are exposed to aqueous solution. The unfavorable interactions that may occur between the bulk aqueous phase and the non-polar tail groups (220) are further reduced when the planar bi-layer sheets fold on themselves to form closed sealed vesicles containing an enclosed aqueous compartment. Unilamellar vesicles are formed from one such bilayer and multilamellar vesicles have multiple concentric bilayers.

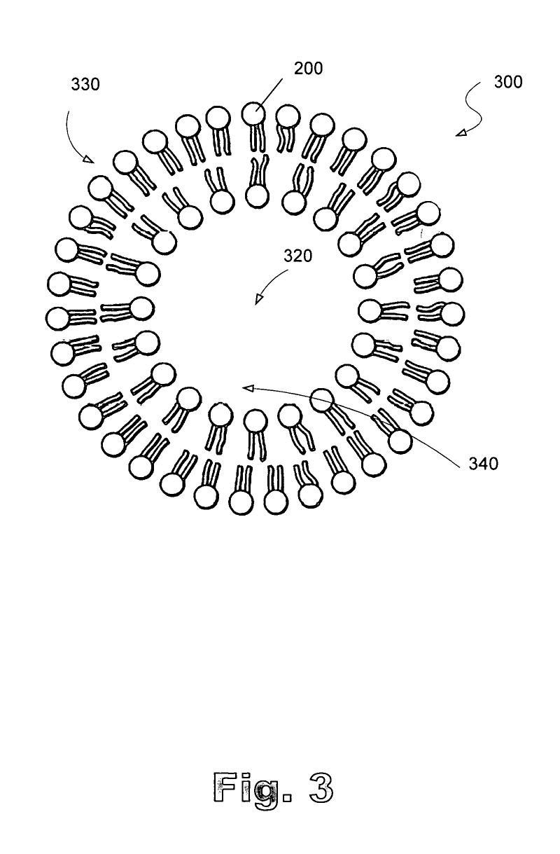

FIG. 3 illustrates a closed sealed unilamellar vesicle (300) according to one exemplary embodiment. As illustrated in FIG. 3, when in an aqueous solution, a plurality of lipids (200) form a multilayered membrane of lipid (200) molecules, each molecule having non-polar (220) and polar ends (210). According to the unilamellar vesicle (300) illustrated in FIG. 3, the polar ends (210) of the lipids (200) form the exterior surface of the unilamellar vesicle (300) while the non-polar ends (220) form the inner structure of the outer membrane (330).

The multilayered vesicle (300) structures of vesicle-forming lipids tend to form in preference to micellar structures because the two non-polar groups tend to impart to the molecule an overall tubular shape, which is more suitable for this type of aggregation. According to the exemplary embodiment illustrated in FIG. 3, the unilamellar liposome vesicle (300) includes an aqueous cavity (320) configured to entrap materials both within the inner compartment of the aqueous cavity and between the layers of the inner (340) and outer (330) membranes. According to one exemplary embodiment illustrated in FIG. 3, the unilamellar vesicle (300) is configured to protect and confine the entrapped material until the vesicle (300) adheres to the outer membrane of a target cell. Consequently, when the vesicle-forming lipids are applied to a pharmaceutical delivery application, drug efficacy may be increased while overall toxicity is reduced due to the direct delivery of the pharmaceutical to the needed cells.

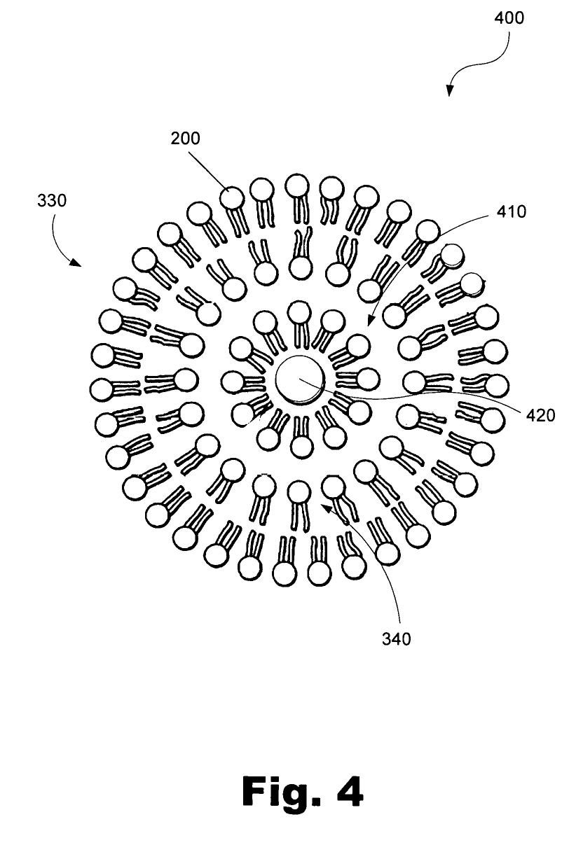

In addition, as illustrated in FIG. 4, some liposome vesicles are multilamellar vesicles (400) rather than unilamellar. As illustrated in FIG. 4, a multi lamellar vesicle (400) may be formed including a first membrane having an outer membrane (330) and an inner membrane (340) configured as described above with reference to FIG. 3. However, as illustrated in FIG. 4, a second membrane (410) may also be concentrically formed within the outer membrane. As illustrated in FIG. 4, a pharmaceutical payload (420) may be entrapped within the second membrane (410). Additionally, a pharmaceutical payload (420) may be entrapped between the various membranes. Alternatively, a pharmaceutical payload (420) can be surrounded by or associated with one or more liposome vesicles, with interactions such as adsorption, solution, Van der Waals and charged or ionic. In one embodiment, the pharmaceutical may be integral part of the vesicle structure. In yet another embodiment, both the pharmaceutical payload (420) and the liposome vesicles can be present in a solution though not physically associated with each other. The exemplary composition of the aqueous vesicle pharmaceutical (160; FIG. 1) will now be described in further detail below.

Exemplary Composition

According to one exemplary embodiment, the present aqueous vesicle pharmaceutical (160; FIG. 1) includes an edible aqueous vehicle component that may or may not include a co-solvent, an edible vesicle forming component, and an edible pharmaceutical payload component. Exemplary embodiments of the aqueous vesicle pharmaceutical components, as well as additional additives, are described below.

As noted above, the present aqueous vesicle pharmaceutical (160; FIG. 1) includes a vesicle forming component configured to form multilamellar vesicles (MLVs) or unilamellar vesicles (ULVs) when under the influence of ultrasound or other high shearing devices. According to one exemplary embodiment, the vesicle forming component of the present system and method may be any food and drug administration (FDA) approved liposome system including, but in no way limited to, synthetic and natural lipids such as phosphatidylcholines, phosphatidylethanolamines, phosphatidic acids, phosphatidylserines, phosphatidylglycerols, cardiolipins, poly(ethylene glycol) lipid conjugates, sphingomyelins, cationic lipids, and miscellaneous lipids such as trioctanoin, triolein, dioctanoyl glycerol, cholesterol (ovine wool), lipid A (salmonella minnesota), purified lipid A, and dioleoyl-glutaric acid. Exemplary lipids of the above-mentioned classifications include, but are in no way limited to, dilauroyl, dimyristoyl, dipalmitoyl, distearoyl, diarachidoyl, dioleoyl, dilinoleoyl, dierucoyl, palmitoyl-oleoyl, tetramyrisoyl, tocopherol acid succinate tris salt, soy lecithin, standard lipids, tocopherol succinate, tris(hydroxymethyl)amino methane, 2-amino-ethyl-1,3 propane diol, phosphatidycholines, phosphatidic acids, sphingolipids, glycolipids, gangliosides, cerebrosides, polyethylene glycol esters, ethers of fatty acids, soybean phosphatidylcholines, egg yolk phosphatidylcholines, and appropriate mixtures thereof. These and additional appropriate vesicle forming components may also be acquired from the Avanti Polar Lipids, Inc.

According to one exemplary embodiment, the vesicle forming component comprises between 1 and approximately 30 percent by weight of the final aqueous vesicle pharmaceutical (160; FIG. 1) solution. Preferred amounts of the vesicle forming component are such that the ratio of pharmaceutical payload component to vesicle forming component is between, but is in no way limited to, about 2:1 and about 3:1 by weight.

The pharmaceutical payload component of the present aqueous vesicle pharmaceutical (160; FIG. 1) is a finely ground pharmaceutical particle receptive to encapsulation by a vesicle forming component. According to one exemplary embodiment, the pharmaceutical payload component is pre-processed to a size of less than 10 micron dimensions. Additionally, the pharmaceutical payload component may take the form of any number of immiscible and non-immiscible pharmaceutical products including, but in no way limited to, Quinidex, Procainamide, Verapamil, Nitroglycerin, Quinidine, Calan, Disopyramide, Sotalol, Mexitil, Pindolol, Isosorbide 5-mononitrate, Cordarone, Digoxin, Nifedipine, Timolol, Dihydropyridine, Ethmozine, Rythmol, Acebutolol, Penbutolol, Nadolol, Diltiazem, Carteolol, Tambocor, Nicardipine, Captopril, Bepridil, Felodipine, Isradipine, Enalapril, Vasotec, Enalaprilat, Zestril, Esmolol, Univasc, Accupril, Quinapril, Lotensin, Benazepril, Altace, Trandolapril, Amlodipine, Monopril, Fosinopril, Moexipril, Corvert, and/or derivatives thereof. Further examples of pharmaceutical encapsulation in vesicle structures can be found in Liposome Technology: Entrapment of Drugs and Other Materials. Vol. 2, published by CRC, Boca Raton, Fla. in 1993 and Liposomes in Drug Delivery, published by Harwood Acad. Publ., Yverdon, Switzerland in 1993, both of which are incorporated herein by reference in their entirety.

The aqueous vehicle component of the present system and method is included in the present aqueous vesicle pharmaceutical (160; FIG. 1) for stable dispersion and transport of the pharmaceutical payload component contained within the vesicle forming component as well as any other additives. The aqueous vehicle imparts a jettable viscosity to the aqueous vesicle pharmaceutical (160; FIG. 1) while evaporating at a rate sufficient to make a dispensed dosage resistant to smudging soon after it is deposited. Additionally, as noted previously, the aqueous vehicle component may or may not include a solvent. According to one exemplary embodiment, the aqueous vehicle comprises water. In addition to having a low cost, water is effective as a solvent for many additives, greatly reduces inkjet dispenser compatibility issues, effectively suspends oral drug formulations and colorants, and effectively controls drying rates of the aqueous vesicle pharmaceutical. In another exemplary embodiment, the aqueous vehicle component of the present aqueous vesicle pharmaceutical (160; FIG. 1) includes a mixture of water and an edible alcohol, such as ethyl alcohol. The addition of an alcohol to the aqueous vehicle component affects the viscosity and drying rate of the aqueous vesicle pharmaceutical while also acting as a surfactant.

In addition to the above-mentioned components of the present aqueous vesicle pharmaceutical, a number of additives may be employed to optimize the properties of the ink composition for specific applications. For example, as is well-known to those skilled in the art, biocides may be used in the ink composition to inhibit growth of microorganisms. Other known additives such as viscosity modifiers, humectants, antifoaming agents, surface tension adjusting agents, rheology adjusting agents, pH adjusting agents, drying agents and other acrylic or non-acrylic polymers may be added to improve various properties of the ink compositions as desired.

According to a first exemplary formulation, the present aqueous vesicle pharmaceutical includes approximately 25% vehicle by volume, approximately 2% vesicle forming component by volume, 3 to 6% pharmaceutical payload by volume, and the remainder water.

According to a second exemplary formulation, the present aqueous vesicle pharmaceutical includes approximately 3.54% vitamin E-succinate by volume, 0.812% Tris by volume, 75.64% water by volume, and approximately 20% Diethylene glycol by volume.

According to a third exemplary formulation, the present aqueous vesicle pharmaceutical includes approximately 5% 1,3propanediol by volume, 3% Brij30 by volume, 0.15% hexadecyltrimethylammonium bromide (HTAB) by volume, 1% Cholesterol by volume, 5 to 10% pharmaceutical payload by volume, and the remainder water.

According to a fourth exemplary formulation, the present aqueous vesicle pharmaceutical includes approximately 2.5% egg yolk or Phosphotidyl choline Soy Lecithin by volume, 1.0% Cholic acid Na salt by volume, 5% Diethylene glycol by volume, 5% pharmaceutical payload by volume, and the remainder water.

According to a fifth exemplary formulation, the present aqueous vesicle pharmaceutical includes approximately 5% sucrosemono/di stearate (Crodesta F50) by volume, 5% 1,3 propane diol by volume, 5% pharmaceutical payload by volume, and the remainder water.

While a number of exemplary formulations for the present aqueous vesicle pharmaceutical are given above, they are in no way meant to limit the present system. Rather, they are presented for exemplary purposes only.

Exemplary Implementation and Operation

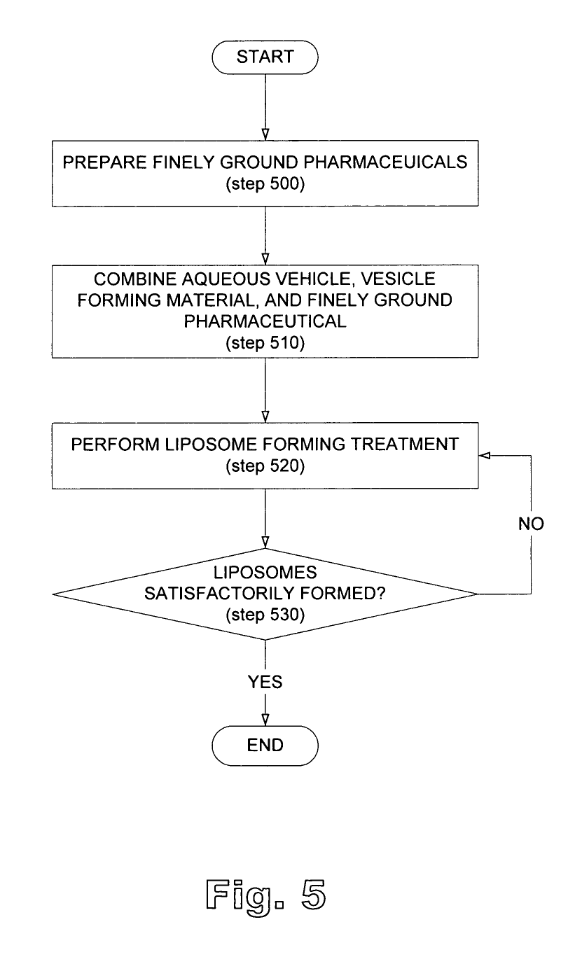

FIG. 5 illustrates and exemplary method for the formation of the aqueous vesicle pharmaceutical (160; FIG. 1) according to one exemplary embodiment. As illustrated in FIG. 5, the formation method begins by preparing a desired quantity of finely ground pharmaceuticals (step 500). Once the finely ground pharmaceuticals are prepared, they are combined with an aqueous vehicle and a vesicle forming material as explained above (step 510). Once the materials are combined, a liposome forming treatment is performed on the combination of materials (step 520). The liposome forming treatment may be checked for satisfactory formation during or after the liposome forming treatment (step 530). The above-mentioned methods will now be explained in further detail below.

As shown in FIG. 5, the present formation method begins by preparing a desired quantity of finely ground pharmaceuticals (step 500). The desired pharmaceuticals may be finely ground according to any number of mechanical or chemical grinding means. According to one exemplary embodiment, the pharmaceuticals are ground by a microfluidizer. According to this exemplary embodiment, the microfluidizer is first used to grind the desired pharmaceutical particles to the appropriate size using a grinding liquid, which may be water or may additionally include one or more edible water-miscible organic solvents. Although not necessary, according to one exemplary embodiment, the grinding liquid is a component of the final aqueous vesicle pharmaceutical composition.

Once the finely ground pharmaceuticals are prepared, they may be combined with an aqueous vehicle and a vesicle forming material (step 510). The finely ground pharmaceuticals, the aqueous vehicle, and the vesicle forming material may be combined into any number of containers using a manual or automated means. Additionally, the combination of the finely ground pharmaceuticals, the aqueous vehicle, and the vesicle forming material may be facilitated by an agitating motion.

Upon mixing the above-mentioned materials, a vesicle forming treatment is performed on the combination (step 520) to form a particle size of less than 200 nm. Any number of vesicle forming treatments may be performed on the combination including, but in no way limited to, mechanical dispersion, micro-emulsification, sonication, membrane extrusion, microfluidization, acute pressure valve homogenization (APV), or the like. A publication that describes many standard materials and techniques relating to the formation of liposome vesicles is Liposome Technology, published by CRC Press in 1993, which is incorporated herein by reference.

According to one exemplary embodiment, the above-mentioned microfluidization method used to reduce the size of the desired pharmaceuticals is extended to the combination of materials in order to form the desired vesicles. The grinding process is continued until the resulting liposome vesicles have a desired mean diameter.

Alternatively, according to a second exemplary embodiment, an APV homogenization method is used to prepare the stable liposome encapsulated materials as described in U.S. Pat. No. 5,976,232 to Gore, et al., which reference is incorporated herein in its entirety. More specifically, according to one exemplary embodiment, the APV treatment enhances print performance of the aqueous vesicle pharmaceutical by producing a solution or ink free of large or agglomerated particles that tend to clog the nozzles of the inkjet material dispenser (150; FIG. 1). Additionally, by producing an aqueous vesicle pharmaceutical solution with a narrow, more uniform size distribution of pigment particles, the stability of the solution is improved.

The APV process by which homogenized aqueous vesicle pharmaceutical solutions are prepared follows herein. According to one exemplary embodiment, the above-mentioned mixture and a “grinding fluid”, typically a dispersant/stabilizer mixture or solvent mixture, is forced under high pressure (from about 10,000 psi to about 30,000 psi) through a valve with small gap and an impact ring (models are commercially available from RANNIE, such as the RANNIE 8.30H, available from APV Homogenizer Group, Wilmington, Mass. 01887.) According to this exemplary embodiment, the particle size of the resulting vesicles is reduced to less than 10 microns. Additionally, the overall range in vesicle particle size is also narrowed, i.e., the vesicle particles on average fall within a more narrow range of sizes.

Depending on the pressure, the vesicle particles, and the grinding fluid mixture employed, the process may be repeated multiple times (anywhere from about 2 to about 100) until a desired size is achieved. While not intending to be bound by any theory, it is believed that the high pressure differential between the inlet of the homogenizer valve and the outlet effects high shear and cavitation in the fluid which alters the size and/or solubility properties of the vesicle particles. It is believed that any conventional homogenizer valve can be used in the practice of this invention as long as the solution that enters the valve is under high enough pressure.

Additionally, the liposome forming treatment may be periodically interrupted to determine if the desired aqueous vesicle pharmaceutical has been satisfactorily formed (step 530). It has been found that certain commercial, high precision filters can be used to verify that an acceptable level of particle size has been achieved, thereby ensuring improved print performance. In contrast to other conventional, commercially available filters, high precision nylon filters, such as those available from Micron Separations Inc. Westborough, Mass., can be used to accurately measure the presence of large particles in the solution. Further, it has been found that the ease of filtration of the ink directly relates to the performance of the solution when dispensed by the inkjet material dispenser (150; FIG. 5). The term “ease” is meant that the number of these filters used to filter a set volume of ink directly relates to the performance of the ink. In other words, if fewer filters are needed to filter a volume of ink, it is traditionally taken as an accurate predictor that the ink will not clog the printer components, especially the printer nozzle).

Once the aqueous vesicle pharmaceutical has been satisfactorily formed, it will exhibit a number of desirable properties. According to one exemplary embodiment, the formed aqueous vesicle pharmaceutical will be suitable for inkjet printing from an inkjet material dispenser (150; FIG. 1). According to this exemplary embodiment, the resulting aqueous vesicle pharmaceutical has a viscosity that is no more than approximately 5 centipoise, although the value may be outside of this range. In addition, the surface tension of the final composition is typically between about 25 to about 60 dynes per centimeter, and more preferably between about 35 to about 50 dynes per centimeter.

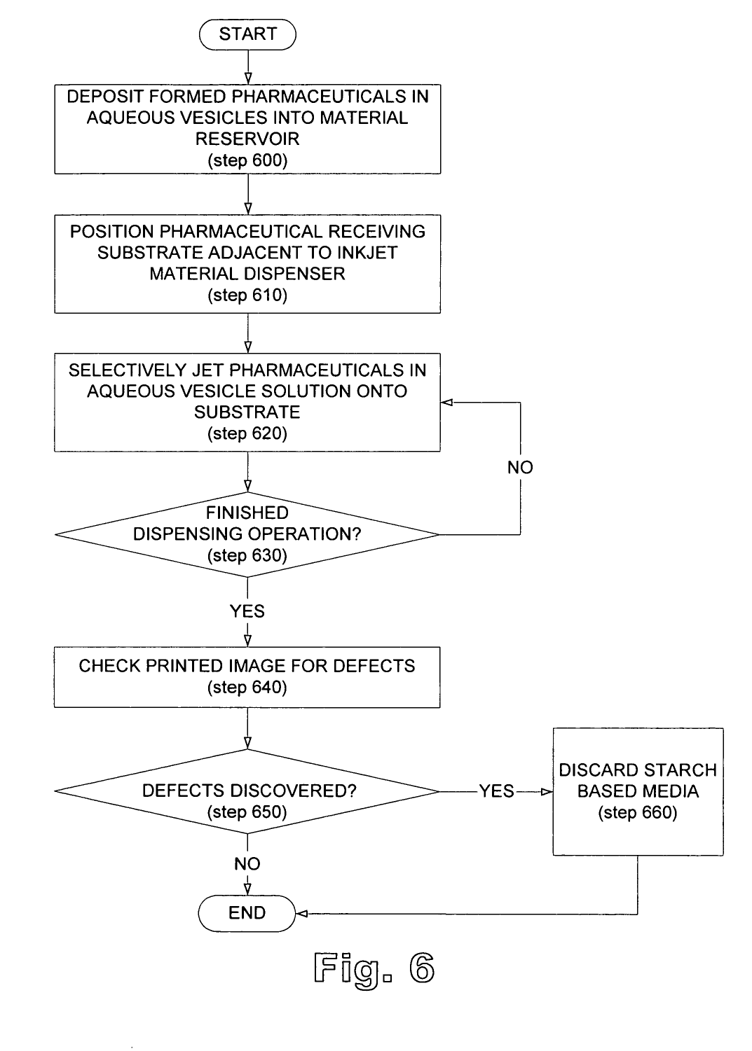

Once the above-mentioned aqueous vesicle pharmaceutical (160; FIG. 1) is formed, it may be selectively jetted onto an edible structure (170; FIG. 1) or other substrate to form a solid drug dosage. FIG. 6 illustrates an exemplary method for jetting an aqueous vesicle pharmaceutical onto an edible structure according to one exemplary embodiment. As shown in FIG. 6, the present method begins by depositing the formed aqueous vesicle pharmaceutical into the material reservoir of a formulation system (step 600). Once the aqueous vesicle pharmaceutical is deposited, an edible structure (170; FIG. 1) is positioned adjacent to the inkjet material dispenser (150; FIG. 1) of the present formulation system (step 610). When positioned, the inkjet material dispenser (150; FIG. 1) selectively deposits the aqueous vesicle pharmaceutical (160; FIG. 1) onto the edible structure (step 620). Upon deposition of the aqueous vesicle pharmaceutical onto the edible structure, a determination is made as to whether the present formulation system (100; FIG. 1) has completed its formulation dispensing operation (step 630). If it is determined that the pharmaceutical formulation dispensing is not complete (NO, step 630), the formulation system again selectively jets an aqueous vesicle pharmaceutical onto the edible structure (step 620). If, however, the pharmaceutical dispensing operation is complete (YES, step 630), the printed media is optionally examined for defects (step 640). If no defects are found (NO, step 450), the aqueous vesicle pharmaceutical dispensing process is complete. If, however, printing defects are found on the printed media (YES, step 650), the edible structure may be discarded (step 660) or otherwise re-processed. The above-mentioned steps will now be described in further detail below.

As shown in FIG. 6, the present method for printing an aqueous vesicle pharmaceutical on an edible structure begins by depositing the formed aqueous vesicle pharmaceutical into a material reservoir (step 600). The deposition of the aqueous vesicle pharmaceutical into a material reservoir may be performed by a user or alternatively by a fluid channeling system disposed between the aqueous vesicle pharmaceutical forming apparatus and the formulation system (100; FIG. 1).

After the formed aqueous vesicle pharmaceutical is deposited into a material reservoir (step 600), an edible structure is positioned adjacent to the inkjet material dispenser (150; FIG. 1) of the present formulation system (step 610). As shown in FIG. 1, the edible structure (170) may be positioned under the formulation system (100) by a moveable substrate (180). Alternatively, an operator or a number of mechanical transportation apparatuses may manually place the edible structure (170) adjacent to the formulation system (100).

Once the edible structure (170) is correctly positioned, the present formulation system (100) may be directed by the computing device (110) to selectively jet the aqueous vesicle pharmaceutical (160) onto the edible structure (step 620; FIG. 6). As was mentioned previously, the desired dosage of the aqueous vesicle pharmaceutical to be printed on the edible structure (170) may initially be determined on a program hosted by the computing device (110). The program created dosage may then be converted into a number of processor accessible commands, which when accessed, may control the servo mechanisms (120) and the movable carriage (140), causing them to selectively emit a specified quantity of aqueous vesicle pharmaceutical (160) onto the edible structure (170).

The precise metering capability of the inkjet material dispenser (150) along with the ability to selectively emit the metered quantity of aqueous vesicle pharmaceutical (160) onto precise, digitally addressed locations makes the present system and method well suited for a number of pharmaceutical delivery applications. According to one exemplary embodiment, the precision and addressable dispensing provided by the present inkjet material dispenser (150) allows for one or more compositions to be dispensed on a single edible structure (170). According to this exemplary embodiment, a combination therapy may be produced in a customized dosage for a patient. Precision of the resulting oral drug deposition may be varied by adjusting a number of factors including, but in no way limited to, the type of inkjet material dispenser (150) used, the distance between the inkjet material dispenser (150) and the edible structure (170), and the dispensing rate. Once the aqueous vesicle pharmaceutical (160) has been selectively deposited onto the edible structure (170), according to the desired dosage, the deposited aqueous vesicle pharmaceutical may be absorbed by the edible structure or remain in a fixed state on top of the edible structure. Consequently, the aqueous vesicle pharmaceutical is affixed to the edible structure until consumption initiates a selective release thereof.

Upon deposition of the aqueous vesicle pharmaceutical, it is determined whether or not the aqueous vesicle pharmaceutical dispensing operation has been completed on the edible structure (step 630; FIG. 6). Completion of the aqueous vesicle pharmaceutical dispensing operation may be evaluated by a system operator or by the coupled computing device (110). According to one exemplary embodiment, the computing device (110) determines whether sufficient aqueous vesicle pharmaceutical (160) has been dispensed to produce the desired dosage on the edible structure (170). If sufficient aqueous vesicle pharmaceutical (160) has not been dispensed (NO, step 630; FIG. 6), the formulation system (100) continues to selectively deposit jetted aqueous vesicle pharmaceutical onto the edible structure (step 620; FIG. 6). If, however, sufficient aqueous vesicle pharmaceutical (160) has been dispensed (YES, Step 630; FIG. 6), the dispensed quantity may optionally be checked for defects (step 640; FIG. 6).

In order to check the printed media for defects (step 640; FIG. 6), according to one exemplary embodiment, the edible structure (170) or other image receiving substrate may be analyzed according to weight, volume, or optical properties for obvious defects that may make the resulting substrate unacceptable. According to one exemplary embodiment, the edible structure (170) is subject to a series of optical scans configured to detect any alignment or deposition defects. Additionally, adequacy of the volume of aqueous vesicle pharmaceutical (160) dispensed onto an edible structure (170) may be evaluated by a number of flow-rate sensors (not shown) disposed on the inkjet material dispenser (150).

According to one exemplary embodiment, if defects are discovered on the edible structure (YES, step 650; FIG. 6), the edible structure may be discarded (step 660; FIG. 6) and the system adjusted. If, however, no image defects are discovered (NO, step 650; FIG. 6) the edible structure (170) may be packaged or otherwise distributed. According to one exemplary embodiment, the step of packaging and/or otherwise distributing the edible structure (170) may include a number of processes including, but in no way limited to, slicing or otherwise dividing a large edible structure into smaller individual dosages, hermetically sealing the individual dosages, labeling the dosages, and/or packaging the individual dosages.

### Alternative Embodiment

According to one alternative embodiment, the above-mentioned system and method may be performed using a polymersome based aqueous vesicle pharmaceutical. According to this exemplary embodiment, the edible vesicle forming component of the aqueous vesicle pharmaceutical is an edible polymersome made from di-block copolymers. The di-block copolymers may include, but are in no way limited to, polyethyleneoxide-polyethylethylene. According to this alternative embodiment, the resulting polymersome based aqueous vesicle pharmaceutical will exhibit varied characteristics when compared to the liposome based vesicles mentioned above. According to one exemplary embodiment, the polymersome based aqueous vesicle pharmaceutical will have a higher molecular weight and be less permeable to water than the liposome based vesicles, thereby modifying the resulting pharmaceutical release rate.

In conclusion, the present system and method for producing and dispensing an ink jettable aqueous vesicle containing a pharmaceutical payload allows for precision dispensing of insoluble or low-solubility pharmaceuticals. More specifically, the insoluble or low-solubility pharmaceuticals are encapsulated by liposome or polymersome compositions capable of being dispensed by an inkjet material dispenser. Moreover, the use of an inkjet material dispenser allows a high precision of dosage forms. In addition, the disclosed aqueous vesicle pharmaceuticals exhibit a number of desirable properties such as excellent jettability, stability, uniform drop formation, fine particle size, ability to form individual, gel-drops of nanometer size, and precise control over the dosage amount. Additionally, the systems and methods disclosed are cost effective when compared to traditional formulation methods while being able to precisely deliver and prepare custom dosages without special treatments, modifications, or use of special equipment.

The preceding description has been presented only to illustrate and describe exemplary embodiments of the present system and method. It is not intended to be exhaustive or to limit the system and method to any precise form disclosed. Many modifications and variations are possible in light of the above teaching. It is intended that the scope of the system and method be defined by the following claims.

1. A jettable solution comprising:
a plurality of vesicles; and
a pharmaceutical payload associated with said vesicles.

2. The jettable solution of claim 1, wherein said jettable solution further comprises an edible vehicle, said plurality of vesicles being stably dispersed in said edible vehicle.

3. The jettable solution of claim 2, wherein said edible vehicle comprises one of water or an alcohol.

4. The jettable solution of claim 3, wherein sad edible vehicle further comprises a solvent.

5. The jettable solution of claim 1, wherein said plurality of vesicles are formed from a lipid or a mixture of lipids selected from the group consisting of phosphatidylcholines, phosphatidylethanolamines, phosphatidic acids, phosphatidylserines, phosphatidylglycerols, cardiolipins, poly(ethylene glycol) lipid conjugates, sphingomyelins, cationic lipids, trioctanoin, triolein, dioctanoyl glycerol, cholesterol (ovine wool), lipid A (salmonella minnesota), purified lipid A, and dioleoyl-glutaric acid.

6. The jettable solution of claim 1, wherein said plurality of vesicles are formed from a plurality of di-block copolymers.

7. The jettable solution of claim 1, wherein said plurality of vesicles comprise dimensions of less than 10 microns.

8. The jettable solution of claim 1, wherein said pharmaceutical payload comprises a substantially water-insoluble pharmaceutical.

9. The jettable solution of claim 8, wherein said pharmaceutical payload is selected from the group consisting of Quinidex, Procainamide, Verapamil, Nitroglycerin, Quinidine, Calan, Disopyramide, Sotalol, Mexitil, Pindolol, Isosorbide 5-mononitrate, Cordarone, Digoxin, Nifedipine, Timolol, Dihydropyridine, Ethmozine, Rythmol, Acebutolol, Penbutolol, Nadolol, Diltiazem, Carteolol, Tambocor, Nicardipine, Captopril, Bepridil, Felodipine, Isradipine, Enalapril, Vasotec, Enalaprilat, Zestril, Esmolol, Univasc, Accupril, Quinapril, Lotensin, Benazepril, Altace, Trandolapril, Amlodipine, Monopril, Fosinopril, Moexipril, and Corvert.

10. The jettable solution of claim 1, further comprising a property enhancing agent.

11. The jettable solution of claim 10, wherein said property enhancing agent comprises one of a biocide, a viscosity modifier, a humectant, an antifoaming agent, a surface tension adjusting agent, a rheology adjusting agent, a pH adjusting agent, a drying agent, or a polymer.

12. The jettable solution of claim 1, wherein said solution comprises a viscosity of less than 5 centipoise.

13. The jettable solution of claim 1, wherein said solution comprises a surface tension between approximately 25 and 60 dynes per centimeter.

14. The jettable solution of claim 1, wherein said solution is configured to be selectively emitted from an inkjet material dispenser.

15. The jettable solution of claim 14, wherein said inkjet material dispenser comprises one of a thermally actuated inkjet dispenser, a mechanically actuated inkjet dispenser, an electro-statically actuated inkjet dispenser, a magnetically actuated dispenser, a piezo-electrically actuated inkjet dispenser, or a continuous inkjet dispenser.

16. The jettable solution of claim 1, further comprising:
approximately 25% vehicle;
approximately 2% vesicle forming component;
approximately 3 to 6% pharmaceutical payload; and
water.

17. The jettable solution of claim 1, further comprising:
approximately 3.54% vitamin E-succinate;
approximately 0.8% Tris;
approximately 75.64% water; and
approximately 20% Diethylene glycol.

18. The jettable solution of claim 1, further comprising:
approximately 5% 1,3propanediol;
approximately 3% Brij30;
approximately 0.15% hexadecyltrimethylammonium bromide (HTAB);
approximately 1% Cholesterol;
between 5 and 10% pharmaceutical payload; and
water.

19. The jettable solution of claim 1, further comprising:
approximately 2.5% egg yolk or Phosphotidyl choline Soy Lecithin;
approximately 1.0% Cholic acid Na salt;
approximately 5% Diethylene glycol;
approximately 5% pharmaceutical payload; and
water.

20. The jettable solution of claim 1, further comprising:
approximately 5% sucrosemono/di stearate;
approximately 5% 1,3 propane diol;
approximately 5% pharmaceutical payload; and
water.

21. A method for forming a jettable pharmaceutical solution comprising:
presenting a pharmaceutical combining said pharmaceutical with a vesicle forming material and an aqueous vehicle; and
processing said combination to form a jettable solution including a plurality of vesicles containing said pharmaceutical.

22. The method of claim 21, further comprising grinding said pharmaceutical to a particle size of less than 200 nanometers.

23. The method of claim 22, wherein said grinding further comprises processing said pharmaceutical with a microfluidizer.

24. The method of claim 21, wherein said pharmaceutical comprises a substantially water insoluble pharmaceutical.

25. The method of claim 21, wherein said pharmaceutical is selected from the group consisting of Quinidex, Procainamide, Verapamil, Nitroglycerin, Quinidine, Calan, Disopyramide, Sotalol, Mexitil, Pindolol, Isosorbide 5-mononitrate, Cordarone, Digoxin, Nifedipine, Timolol, Dihydropyridine, Ethmozine, Rythmol, Acebutolol, Penbutolol, Nadolol, Diltiazem, Carteolol, Tambocor, Nicardipine, Captopril, Bepridil, Felodipine, Isradipine, Enalapril, Vasotec, Enalaprilat, Zestril, Esmolol, Univasc, Accupril, Quinapril, Lotensin, Benazepril, Altace, Trandolapril, Amlodipine, Monopril, Fosinopril, Moexipril, and Corvert.

26. The method of claim 21, wherein said vesicle forming material comprises a plurality of lipids.

27. The method of claim 26, wherein said plurality of lipids are selected from the group consisting of phosphatidylcholines, phosphatidylethanolamines, phosphatidic acids, phosphatidylserines, phosphatidylglycerols, cardiolipins, poly(ethylene glycol) lipid conjugates, sphingomyelins, cationic lipids, trioctanoin, triolein, dioctanoyl glycerol, cholesterol (ovine wool), lipid A (salmonella minnesota), purified lipid A, and dioleoyl-glutaric acid.

28. The method of claim 21, wherein said vesicle forming material comprises a plurality of di-block copolymers.

29. The method of claim 28, wherein said di-block copolymers comprise polylethyleneoxide-polyethylethylene.

30. The method of claim 21, wherein said aqueous vehicle comprises one of water or an alcohol.

31. The method of claim 30, wherein said aqueous vehicle further comprises a solvent.

32. The method of claim 21, wherein said combining said pharmaceutical with a vesicle forming material and an aqueous vehicle comprises mixing said pharmaceutical, said vesicle forming material, and said aqueous vehicle.

33. The method of claim 21, wherein said processing said combination to form a jettable solution including a plurality of vesicles containing said pharmaceutical comprises performing one of a mechanical dispersion process, a micro-emulsification process, a sonication process, a membrane extrusion process, a microfluidization process, or an acute pressure valve homogenization (APV) process.

34. The method of claim 33, wherein said APV homogenization process comprises:
forcing said combination through a valve having a small orifice and an impact ring.

35. The method of claim 21, wherein said jettable solution is configured to be selectively emitted from an inkjet material dispenser.

36. The method of claim 35, wherein said inkjet material dispenser comprises one of a thermally actuated inkjet dispenser, a mechanically actuated inkjet dispenser, an electro-statically actuated inkjet dispenser, a magnetically actuated dispenser, a piezo-electrically actuated inkjet dispenser, or a continuous inkjet dispenser.

37. The method of claim 21, wherein said plurality of vesicles comprise a dimension of less than 10 microns.

38. The method of claim 37, wherein said jettable solution comprises a viscosity of less than 5 centipoise.

39. The method of claim 37, wherein said jettable solution comprises a surface tension between approximately 25 and 60 dynes per centimeter.

40. The method of claim 21, further comprising dispensing a property enhancing agent into said combination.

41. The method of claim 40, wherein said property enhancing agent comprises one of a biocide, a viscosity modifier, a humectant, an antifoaming agent, a surface tension adjusting agent, a rheology adjusting agent, a pH adjusting agent, a drying agent, or a polymer.

42. A method for forming an oral pharmaceutical comprising:
presenting an edible structure adjacent to an inkjet material dispenser; and
selectively dispensing an aqueous vesicle pharmaceutical from said inkjet material dispenser onto said edible structure.

43. The method of claim 42, wherein said inkjet material dispenser comprises one of a thermally actuated inkjet dispenser, a mechanically actuated inkjet dispenser, an electrostatically actuated inkjet dispenser, a magnetically actuated dispenser, a piezo-electrically actuated inkjet dispenser, or a continuous inkjet dispenser.

44. The method of claim 42, wherein said selectively dispensing comprises dispensing a predetermined dosage of said aqueous vesicle pharmaceutical.

45. The method of claim 42, wherein said edible structure comprises one of a polymeric or paper organic film former.

46. The method of claim 42, wherein said aqueous vesicle pharmaceutical comprises a pharmaceutical payload enclosed within a liposome vesicle.

47. The method of claim 42, further comprising dividing said edible structure into a plurality of single oral doses.

48. The method of claim 42, further comprising selectively dispensing a plurality of aqueous vesicle pharmaceuticals onto said edible structure, said plurality of aqueous pharmaceuticals forming a combination therapy.

49. A system for dispensing an oral solution comprising:
an edible structure; and
a vesicle solution containing a pharmaceutical payload configured to be dispensed onto said edible structure.

50. The system of claim 49, wherein said edible structure comprises one of a rice starch based paper, a potato starch based paper, or an edible polymer.

51. The system of claim 49, wherein said vesicle solution comprises vesicles formed from one of a liposome or a polymersome.

52. The system of claim 49, further comprising:
a computing device disposed adjacent to said edible structure;
an inkjet material dispenser communicatively coupled to said computing device; and
a material reservoir fluidly coupled to said inkjet material dispenser, said material reservoir being configured to supply said liposome vesicle solution containing a pharmaceutical payload to said inkjet material dispenser.

53. The system of claim 52, wherein said computing device comprises one of a personal computer, a laptop computer, a personal digital assistant, or a cellular telephone.

54. The system of claim 52, wherein said inkjet material dispenser comprises one of a thermally actuated inkjet dispenser, a mechanically actuated inkjet dispenser, an electrostatically actuated inkjet dispenser, a magnetically actuated dispenser, a piezo-electrically actuated inkjet dispenser, or a continuous inkjet dispenser.

55. A jettable solution comprising:
a water insoluble pharmaceutical payload; and
a means for encapsulating said pharmaceutical payload into a jettable solution.

56. The jettable solution of claim 55, wherein said jettable solution further comprises a means for stably dispersing said encapsulated pharmaceutical payload.

57. A system for dispensing an oral solution comprising:
an edible means for receiving a pharmaceutical payload solution; and
a liposome vesicle solution containing a pharmaceutical payload configured to be dispensed onto said means for receiving a pharmaceutical payload solution.

58. The system of claim 57, wherein said edible means for receiving a pharmaceutical payload solution comprises one of a rice starch based paper, a potato starch based paper, or an edible polymer.

59. The system of claim 58, further comprising:
a means for computing disposed adjacent to said edible structure;
a means for selectively dispensing said pharmaceutical payload solution communicatively coupled to said means for computing; and
a material reservoir fluidly coupled to said means for selectively dispensing said pharmaceutical payload solution, said material reservoir being configured to supply said pharmaceutical payload solution to said inkjet material dispenser.
